(19) United States
(12) Patent Application Publication
(10) Pub. No.: US 2021/0307366 A1
(43) Pub. Date: Oct. 7, 2021

(54) SOLID DISPERSION COMPOSITION, POWDER PREPARATION AND METHOD FOR PRODUCING SAME, AND FOOD OR BEVERAGE, ETC.

(71) Applicant: SAN-EI GEN F.F.I., INC., Toyonaka-shi, Osaka (JP)

(72) Inventors: Takaaki ITO, Toyonaka-shi, Osaka (JP); Ryosuke NOMURA, Toyonaka-shi, Osaka (JP); Shunsuke KINOSHITA, Toyonaka-shi, Osaka (JP)

(21) Appl. No.: 17/250,512

(22) PCT Filed: Jul. 31, 2019
(86) PCT No.: PCT/JP2019/029987

§ 371 Date: Jan. 29, 2021

(30) Foreign Application Priority Data
Jul. 31, 2018 (JP) 2018-144326

(51) Int. Cl. A23L 2/60; A23L 33/105

(52) U.S. Cl. CPC A23L 2/60; A23L 33/105

(57) ABSTRACT

A solid dispersion composition has excellent stability of lutein and zeaxanthin. The solid dispersion composition contains solid lutein, solid zeaxanthin, gum ghatti, and water. A method for producing a powder preparation includes preparing the solid dispersion composition and powdering the solid dispersion composition. A food or beverage can have the solid dispersion composition or the powder preparation.

### TECHNICAL FIELD

The present invention relates to a solid dispersion composition containing solid lutein, solid zeaxanthin, and gum ghatti, a powder preparation and a method for producing the same, and a food or beverage or the like using the same.

### BACKGROUND ART

Lutein and zeaxanthin are each a type of carotenoid having a strong antioxidant activity, and are contained much in green and yellow vegetables such as kale and spinach. The lutein is a beneficial component in the protection and healing of the retina of the eye, and has a beneficial effect on improving the visual function of the eye and also alleviating neck stiffness and general malaise caused by eye strain, and therefore is also blended in foods and beverages including drinks. Further, recently, foods and beverages (for example, foods with function claims) which appeal to the functionality (for example, physiological activity) of lutein have also been studied.

Heretofore, as lutein-containing foods and beverages, a lutein-containing beverage containing lutein and a blueberry extract as main components (see, for example, PTL 1), a beverage containing lutein and having a pH of 3.8 or more and 4.8 or less (see, for example, PTL 2), and the like have been proposed.

### CITATION LIST

### Patent Literature

PTL 1: JP-A-2005-65525

PTL 2: JP-A-2017-169508

### SUMMARY OF INVENTION

### Technical Problem

The lutein-containing beverage disclosed in PTL 1 uses lutein which is an emulsion. However, a preparation in which lutein is emulsified has a problem that the residual ratio of lutein is decreased due to light (light resistance is insufficient). In addition, in a food or beverage which appeals to the functionality of lutein, it is desirable to specify the content of lutein in the composition, however, an emulsified lutein preparation in the prior art also has a problem that it is difficult to quantitafy the content of lutein.

Further, in the technique disclosed in PTL 2, the pH of the beverage is limited, or the like, and therefore, the technique has low versatility. Further, no attention has been paid to the lutein preparation itself, and no technique for improving light resistance has been studied.

In view of the above prior art, an object to be achieved by the present invention is to provide a solid dispersion composition having excellent stability of lutein and zeaxanthin. More specifically, an object is to provide a solid dispersion composition having excellent stability of lutein and zeaxanthin against light and a solid dispersion composition having excellent dispersion stability of lutein and zeaxanthin after storage or the like.

Further, another object to be achieved by the present invention is to provide a powder preparation having excellent light resistance and dispersion stability of lutein and zeaxanthin when used in a food or beverage or the like.

Further, another object to be achieved by the present invention is to provide a production method for obtaining a powder preparation having excellent light resistance and dispersion stability of lutein and zeaxanthin when used in a food or beverage or the like.

Further, another object to be achieved by the present invention is to provide a food or beverage or the like having excellent light resistance and dispersion stability of lutein and zeaxanthin.

Further, another object to be achieved by the present invention is to provide a solid dispersion composition and a powder preparation capable of quantifying lutein and zeaxanthin, and a food or beverage or the like capable of quantitatively indicating the blending amount of lutein and zeaxanthin.

### Solution to Problem

The present inventors made intensive studies, and as a result, they found that by using a solid dispersion composition containing solid lutein, solid zeaxanthin, gum ghatti, and water, and a powder preparation containing solid lutein, solid zeaxanthin, and gum ghatti, a food or beverage or the like having excellent stability of lutein and zeaxanthin can be obtained, and completed the present invention based on such finding.

The present invention includes aspects described in the following respective items.

Item 1. A solid dispersion composition, containing solid lutein, solid zeaxanthin, gum ghatti, and water.

Item 2. The solid dispersion composition according to Item 1, wherein in the solid dispersion composition, the content ratio (mass % ratio) of the solid lutein to the solid zeaxanthin is from 20:1 to 2:1.

Item 3. The solid dispersion composition according to Item 1 or 2, wherein the average particle diameter of each of the solid lutein and the solid zeaxanthin is from 0.03 to 2 μm.

Item 4. The solid dispersion composition according to any one of Items 1 to 3, wherein in the solid dispersion composition, the total content of the solid lutein and the solid zeaxanthin is from 0.1 to 30 mass %.

Item 5. The solid dispersion composition according to any one of Items 1 to 4, wherein the content of gum ghatti with respect to 1 part by mass of the total content of the solid lutein and the solid zeaxanthin is from 0.1 to 15 parts by mass.

Item 6. The solid dispersion composition according to any one of Items 1 to 5, wherein the pH is less than 5.

Item 7. A method for producing a powder preparation, including:

a step of preparing the solid dispersion composition according to any one of Items 1 to 6; and

a step of powdering the solid dispersion composition.

Item 8. A powder preparation, containing solid lutein, solid zeaxanthin, and gum ghatti.

Item 9. The powder preparation according to Item 8, wherein in the powder preparation, the content ratio (mass % ratio) of the solid lutein to the solid zeaxanthin is from 20:1 to 2:1.

Item 10. The powder preparation according to Item 8 or 9, wherein the average particle diameter of each of the solid lutein and the solid zeaxanthin is from 0.03 to 2 μm.

Item 11. The powder preparation according to any one of Items 8 to 10, wherein the content of gum ghatti with respect to 1 part by mass of the total content of the solid lutein and the solid zeaxanthin is from 0.1 to 15 parts by mass.

Item 12. The powder preparation according to any one of Items 8 to 11, wherein the pH when a 1 mass % aqueous solution of the powder preparation is formed is less than 5.

Item 13. A food or beverage, a coloring agent, a coloring additive, a cosmetic, an aromatic, a body cleanser, a hair care product, a skin care product, an oral product, a bath additive, a pharmaceutical product, or a quasi-drug (hereinafter these are sometimes referred to as “a food or beverage or the like”), containing the solid dispersion composition according to any one of Items 1 to 6 or the powder preparation according to any one of Items 8 to 12.

Item 14. The food or beverage, the coloring agent, the coloring additive, the cosmetic, the aromatic, the body cleanser, the hair care product, the skin care product, the oral product, the bath additive, the pharmaceutical product, or the quasi-drug according to Item 13, wherein the pH is less than 3.8.

### Advantageous Effects of Invention

By using the solid dispersion composition of the present invention, a solid dispersion composition, a powder preparation, and a food or beverage or the like having excellent stability of lutein and zeaxanthin can be obtained. More specifically, for example, a solid dispersion composition having excellent stability against light (light resistance) of lutein and zeaxanthin, a solid dispersion composition, a powder preparation, and a food or beverage or the like having excellent dispersion stability of lutein and zeaxanthin after storage or the like can be obtained.

Further, by using the powder preparation of the present invention, a powder preparation and a food or beverage or the like having excellent light resistance and dispersion stability of lutein and zeaxanthin when it is used in a food or beverage or the like can be obtained.

Further, by using the method for producing a powder preparation of the present invention, the above-mentioned powder preparation having excellent light resistance and dispersion stability of lutein and zeaxanthin when it is used in a food or beverage or the like can be easily obtained.

Further, by using the food or beverage or the like of the present invention, a food or beverage or the like having excellent light resistance and dispersion stability of lutein and zeaxanthin can be obtained.

Further, according to the present invention, a solid dispersion composition and a powder preparation capable of quantifying lutein and zeaxanthin, and a food or beverage or the like capable of quantitatively indicating the blending amount of lutein and zeaxanthin can be obtained.

### BRIEF DESCRIPTION OF DRAWINGS

FIG. 1 shows the results (the residual ratio of lutein and zeaxanthin) of a light resistance test of Test Example 1.

FIG. 2 shows the results of an HPLC analysis of a solid dispersion composition of Example 1 in a quantitative test of Test Example 2.

FIG. 3 shows the results of an HPLC analysis of a lutein and zeaxanthin-containing emulsified preparation (1) in a quantitative test of Test Example 2.

FIG. 4 shows the results of an HPLC analysis of a lutein and zeaxanthin-containing emulsified preparation (2) in a quantitative test of Test Example 2.

FIG. 5 shows a micrographic image of the solid dispersion composition of Example 1 after storage in a storage test of Test Example 3.

FIG. 6 shows a micrographic image of a solid dispersion composition of Comparative Example 2 after storage in a storage test of Test Example 3.

### DESCRIPTION OF EMBODIMENTS

### <Solid Dispersion Composition>

The present invention relates to a solid dispersion composition containing solid lutein, solid zeaxanthin, gum ghatti, and water.

In the present invention, the solid lutein and the solid zeaxanthin refer to a solid in a crystal or crystalline form of each of lutein and zeaxanthin, and also include those which can be added by micronizing a component in a solid form generally usable in a food or beverage or the like. Further, each of the solid lutein and the solid zeaxanthin may be a solid in a single pure crystal or crystalline form, or a solid in a crystal or crystalline form containing both lutein and zeaxanthin may be used.

The solid lutein usable in the present invention is not limited as long as it is a generally available one. Above all, crystalline lutein is preferred, and crystal lutein is particularly preferred.

Note that lutein is a substance obtained as a component derived from a natural product such as an extract from marigold, and has the following structural formula.

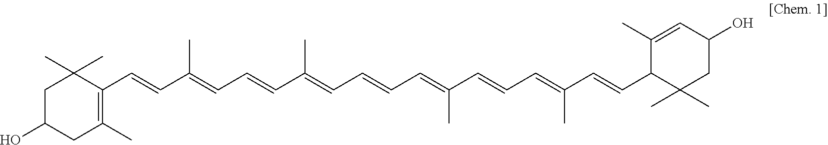 [SMILES: CC1=CC(O)CC(C)(C)C1/C=C/C(C)=C/C=C/C(C)=C/C=C/C=C(C)/C=C/C=C(C)/C=C/C1=C(C)CC(O)CC1(C)C]

Further, the lutein in the present invention includes lutein represented by the above structural formula and a derivative thereof. The derivative includes a compound in which the above chemical structural formula is partially substituted, and a salt thereof. The lutein is desirably lutein represented by the above structural formula.

Further, in the present invention, the term “crystalline” is not limited to one in which a crystal structure is uniformly formed by a repeating unit like a pure crystal, and includes, for example, a crystalline material or a crystal-like solid, and may be any suitable crystal form, and may be one obtained from a natural source or one artificially produced.

The content of the solid lutein in the solid dispersion composition of the present invention can be appropriately changed according to the amounts or types of other components, but is preferably from 0.01 to 30 mass % in the composition, and may be from 0.02 to 25 mass %, or from 0.03 to 20 mass %, or from 0.05 to 18 mass %, or from 0.05 to 15 mass %, or from 0.05 to 12 mass %, or from 0.1 to 10 mass %, or from 0.2 to 8 mass %, or from 0.3 to 5 mass %, or from 0.3 to 3 mass %, or from 0.5 to 3 mass %, or from 0.5 to 2.5 mass %. When the content is the above-mentioned content, a composition having excellent stability of lutein is more reliably formed.

In the present invention, the average particle diameter of the solid lutein is preferably from 0.03 to 2 μm, and may be from 0.03 to 1.5 μm, or from 0.05 to 1.2 μm, or from 0.05 to 1.0 μm, or from 0.08 to 1.0 μm, or from 0.05 to 0.8 μm, or from 0.05 to 0.6 μm, or from 0.05 to 0.5 μm, or from 0.1 to 0.6 μm, or from 0.1 to 0.5 μm. By setting the average particle diameter to the above-mentioned average particle diameter, a composition having excellent stability of lutein is more reliably formed.

The solid zeaxanthin usable in the present invention is not limited as long as it is a generally available one. Above all, crystalline zeaxanthin is preferred, and crystal zeaxanthin is particularly preferred.

Note that zeaxanthin is a substance obtained as a component derived from a natural product such as an extract from marigold, and has the following structural formula.

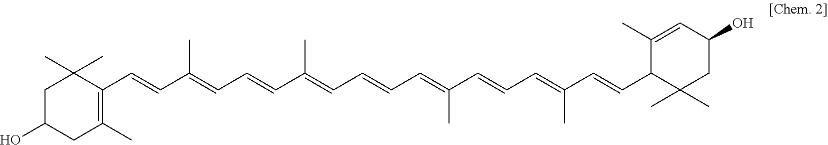 [SMILES: CC1=C[C@@H](O)CC(C)(C)C1/C=C/C(C)=C/C=C/C(C)=C/C=C/C=C(C)/C=C/C=C(C)/C=C/C1=C(C)CC(O)CC1(C)C]

Further, the zeaxanthin in the present invention includes zeaxanthin represented by the above structural formula and a derivative thereof. The derivative includes a compound in which the above chemical structural formula is partially substituted, and a salt thereof. The zeaxanthin is desirably zeaxanthin represented by the above structural formula.

The content of the solid zeaxanthin in the solid dispersion composition of the present invention can be appropriately changed according to the amounts or types of other components, but is preferably from 0.001 to 15 mass % in the composition, and may be from 0.002 to 13 mass %, or from 0.003 to 10 mass %, or from 0.005 to 10 mass %, or from 0.005 to 7 mass %, or from 0.005 to 6 mass %, or from 0.01 to 5 mass %, or from 0.02 to 4 mass %, or from 0.03 to 3 mass % or from 0.03 to 2 mass %, or from 0.05 to 2 mass %, or from 0.05 to 1 mass %, or from 0.05 to 0.5 mass %, or from 0.05 to 0.4 mass %, or from 0.06 to 0.4 mass %, or from 0.05 to 0.3 mass %, or from 0.06 to 0.3 mass %, or from 0.08 to 0.3 mass %. When the content is the above-mentioned content, a composition having excellent stability of zeaxanthin is more reliably formed.

In the present invention, the average particle diameter of the solid zeaxanthin is preferably from 0.03 to 2 μm, and may be from 0.03 to 1.5 μm, or from 0.05 to 1.2 μm, or from 0.05 to 1.0 μm, or from 0.08 to 1.0 μm, or from 0.05 to 0.8 μm, or from 0.05 to 0.6 μm, or from 0.05 to 0.5 μm, or from 0.1 to 0.6 μm, or from 0.1 to 0.5 μm. By setting the average particle diameter to the above-mentioned average particle diameter, a composition having excellent stability of zeaxanthin is more reliably formed.

Further, in the present invention, in the solid dispersion composition, the content ratio (mass % ratio) of the solid lutein to the solid zeaxanthin is preferably from 20:1 to 2:1, and the content ratio (mass % ratio) may be from 20:1 to 3:1, or from 20:1 to 4:1, or from 20:1 to 5:1, or from 19:1 to 2:1, or from 18:1 to 2:1, or from 18:1 to 3:1, or from 18:1 to 4:1, or from 18:1 to 5:1, or from 17:1 to 2:1, or from 17:1 to 3:1, or from 17:1 to 4:1, or from 17:1 to 5:1, or from 16:1 to 2:1, or from 15:1 to 2:1, or from 14:1 to 2:1, or from 13:1 to 2:1, or from 12:1 to 2:1, or from 11:1 to 2:1, or from 10:1 to 2:1, or from 9:1 to 2:1, or from 8:1 to 3:1, or from 7:1 to 4:1, or from 6:1 to 4:1, or from 6:1 to 5:1. When the content ratio of lutein to zeaxanthin is the above-mentioned ratio, a food or beverage which appeals to the functionality (for example, physiological activity) of lutein and zeaxanthin can be provided.

Further, in the solid dispersion composition, the total content of the solid lutein and the solid zeaxanthin is preferably from 0.01 to 30 mass %, and may be from 0.02 to 25 mass %, or from 0.03 to 20 mass %, or from 0.05 to 18 mass %, or from 0.05 to 15 mass %, or from 0.05 to 12 mass %, or from 0.1 to 10 mass %, or from 0.2 to 8 mass %, or from 0.3 to 5 mass %, or from 0.3 to 3 mass %, or from 0.5 to 3 mass %, or from 0.5 to 2 mass % in the composition.

The gum ghatti usable in the present invention is not limited as long as it is a generally available one. For example, a water-soluble polysaccharide that is a polysaccharide derived from the sap (secretory fluid) of Anogeissus latifolia Wallich of the family Combretaceae, and that is generally dissolved in water in an amount up to about 30 mass % under the temperature condition of room temperature or higher is exemplified.

For example, as the weight average molecular weight of the gum ghatti, a range of 1.1×10⁶ to 2×10⁶ can be exemplified although not particularly limited thereto in the present invention.

Further, as a commercially available gum ghatti preparation, for example, “Gum Ghatti SD” manufactured by San-Ei Gen F.F.I., Inc. is exemplified.

Further, as the gum ghatti usable in the present invention, for example, low-molecular weight gum ghatti can also be used. The weight average molecular weight of the low-molecular weight gum ghatti is, for example, in the range of 0.020×10⁶ to 0.60×10⁶, and preferably in the range of 0.025×10⁶ to 0.50×10⁶, more preferably in the range of 0.030×10⁶ to 0.40×10⁶, further more preferably in the range of 0.030×10⁶ to 0.30×10⁶, and still further more preferably in the range of 0.040×10⁶ to 0.30×10⁶.

The molecular weight distribution (weight average molecular weight/number average molecular weight ratio) (Mw/Mn) of the low-molecular weight gum ghatti is preferably in the range of 1.1 to 13, more preferably in the range of 1.1 to 10, further more preferably in the range of 1.1 to 8, still more preferably in the range of 1.1 to 6, and particularly preferably in the range of 1.1 to 4.

The molecular weight of the gum ghatti of the present invention and its distribution are measured, for example, by the following method.

### (GPC Analysis Conditions)

Detector: RI

Mobile phase: 100 mM K₂SO₄

Flow rate: 1.0 mL/min

Temperature: 40° C.

Column: TSKgel GMPWXL 30 cm (Guard PWXL)

Injection: 100 μL

Pullulan Standard: Shodex STANDARD P-82

Further, in the solid dispersion composition, the content of the gum ghatti is preferably from 1 to 30 mass %, and may be from 2 to 25 mass %, or from 2.5 to 20 mass %, or from 3 to 15 mass %, or from 3 to 10 mass %, or from 3.5 to 15 mass %, or from 4 to 12 mass %, or from 5 to 10 mass %. By setting the content to the above-mentioned content ratio, a composition having excellent stability of lutein and zeaxanthin is more reliably formed.

Further, in the present invention, in the solid dispersion composition, the content of the gum ghatti with respect to 1 part by mass of the total content of the solid lutein and the solid zeaxanthin is preferably from 0.1 to 15 parts by mass, and may be from 0.5 to 15 parts by mass, or from 1 to 15 parts by mass, or from 0.1 to 12 parts by mass, or from 0.5 to 12 parts by mass, or from 1 to 12 parts by mass, or from 0.1 to 10 parts by mass, or from 0.5 to 10 parts by mass, or from 1 to 10 parts by mass, or from 0.3 to 9 parts by mass, or from 0.5 to 9 parts by mass, or from 0.5 to 8 parts by mass, or from 0.8 to 7 parts by mass, or from 1 to 6 parts by mass. By setting the content to the above-mentioned content ratio, a composition having excellent stability of lutein and zeaxanthin is more reliably formed.

As the water to be used in the present invention, any water usable in a food or beverage or the like can be used without particular limitation. It is preferably water usable in a food or beverage, and is, for example, ion exchanged water.

Further, in the present invention, in the solid dispersion composition, the content of the water is preferably from 10 to 85 mass %, and may be from 10 to 80 mass %, or from 20 to 85 mass %, or from 30 to 85 mass %, or from 50 to 85 mass %, or from 10 to 80 mass %, or from 20 to 80 mass %, or from 40 to 80 mass %, or from 10 to 75 mass %, or from 15 to 75 mass %. By setting the content to the above-mentioned content ratio, a composition according to the intended use can be prepared.

In the present invention, the solid dispersion composition means a composition in which the solid lutein and the solid zeaxanthin are in a dispersed state. The solid dispersion composition may be in the form of, for example, a solid, a paste, a gel, a semi-liquid, a liquid, or the like. The solid dispersion composition of the present invention is a solid, and therefore has an excellent taste as compared with an emulsified preparation.

In the present invention, the solid dispersion composition can be configured to have a pH less than 5. For example, as described in PTL 2, lutein is easily decomposed by dehydroxylation in an acidic condition of pH less than 3.8, and it was difficult to provide a lutein and zeaxanthin preparation that is stable in an acidic condition in the prior art. Further, for example, there was a problem that precipitation or aggregation is caused by acidification, or the like in the prior art.

However, according to the present invention, even when the pH is as low as less than 5, a solid dispersion composition having excellent stability can be provided, and it can be used also in a food or beverage or the like having a lower pH. The pH of the solid dispersion composition may be less than 5, or less than 4.5, or less than 4, or less than 3.8, or less than 3.5, or less than 3.3, or less than 3, and as the lower limit, for example, pH 1, pH 1.5, pH 2, pH 2.5, pH 2.8, or pH 3, or the like can be exemplified. Further, the solid dispersion composition contains water, and therefore, in general, the pH measured as it is can be used, however, when appropriate measurement is difficult as it is, the pH when an aqueous solution having a solid content of 1 mass % is formed is used as a reference. By using the solid dispersion composition of the present invention, for example, even in the case of a food or beverage or the like having a pH less than 3.8 (for example, a food or beverage or the like having a pH in the range where the lower limit is pH 1, pH 1.5, pH 2, pH 2.5, pH 2.8, pH 3, pH 3.3, pH 3.5, pH 3.7, or the like, and the upper limit is pH 3.7, pH 3.6, pH 3.5, pH 3.4, pH 3.3, pH 3.2, pH 3.1, pH 3.0, or the like, which are appropriately combined) which is impossible by the proposal of PTL 2 that the pH is set to a specific value of 3.8 to 4.8, a food or beverage or the like having excellent stability is obtained.

Further, in the solid dispersion composition of the present invention, for example, a pH adjusting agent, a fragrance, a coloring agent, vitamin C (including a derivative), or the like, which is used in a food or beverage or the like, may be appropriately blended other than these.

In addition, an emulsified preparation in the prior art indispensably requires a heating treatment for dissolving lutein and zeaxanthin in an oil phase, and therefore, deterioration such as cis-trans denaturation or thermal deterioration of the lutein or the zeaxanthin may be sometimes caused. However, the solid dispersion composition of the present invention does not require a heating treatment or the like for dissolving lutein and zeaxanthin in an oil phase, or the like, and therefore, the composition can be used without causing deterioration of the lutein or the zeaxanthin.

Further, the solid dispersion composition of the present invention has the above-mentioned configuration, and therefore, the contents of lutein and zeaxanthin can be easily quantified. Accordingly, in a food or beverage using the solid dispersion composition of the present invention, the accurate contents of lutein and zeaxanthin can be specified. This is a great advantage in a food or beverage which appeals to the functionality of lutein and zeaxanthin.

### <Powder Preparation>

The present invention relates to a powder preparation containing solid lutein, solid zeaxanthin, and gum ghatti.

Note that with respect to items that are not particularly described as a powder preparation, basically, the description of each component or the like used in the solid dispersion composition in this specification shall be replaced similarly.

The solid lutein usable in the powder preparation is not limited as long as it is generally available. Above all, crystalline lutein is preferred, and crystal lutein is particularly preferred.

The content of the solid lutein in the powder preparation of the present invention can be appropriately changed according to the amounts or types of other components, but is preferably from 0.01 to 30 mass % in the powder preparation, and may be from 0.02 to 25 mass %, or from 0.03 to 20 mass %, or from 0.05 to 18 mass %, or from 0.05 to 15 mass %, or from 0.05 to 12 mass %, or from 1 to 30 mass %, or from 3 to 25 mass %, or from 5 to 25 mass %, or from 7 to 25 mass %, or from 3 to 20 mass %, or from 4 to 20 mass %, or from 5 to 20 mass %, or from 7 to 20 mass %, or from 8 to 18 mass %, or from 3 to 15 mass %, or from 5 to 15 mass %. When the content is the above-mentioned content, a powder preparation having excellent stability of lutein is more reliably formed.

In the present invention, the average particle diameter of the solid lutein is preferably from 0.03 to 2 μm, and may be from 0.03 to 1.5 μm, or from 0.05 to 1.2 μm, or from 0.05 to 1.0 μm, or from 0.08 to 1.0 μm, or from 0.05 to 0.8 μm, or from 0.05 to 0.6 μm, or from 0.05 to 0.5 μm, or from 0.1 to 0.6 μm, or from 0.1 to 0.5 μm. By setting the average particle diameter to the above-mentioned average particle diameter, a powder preparation having excellent stability of lutein is more reliably formed.

The solid zeaxanthin usable in the powder preparation is not limited as long as it is a generally available one. Above all, crystalline zeaxanthin is preferred, and crystal zeaxanthin is particularly preferred.

The content of the solid zeaxanthin in the powder preparation of the present invention can be appropriately changed according to the amounts or types of other components, but is preferably from 0.001 to 15 mass % in the powder preparation, and may be from 0.002 to 13 mass %, or from 0.003 to 10 mass %, or from 0.005 to 9 mass %, or from 0.005 to 8 mass %, or from 0.005 to 6 mass %, or from 0.1 to 15 mass %, or from 0.3 to 13 mass %, or from 0.5 to 13 mass %, or from 0.7 to 13 mass %, or from 0.3 to 10 mass %, or from 0.4 to 10 mass %, or from 0.5 to 10 mass %, or from 0.7 to 10 mass %, or from 0.8 to 9 mass %, or from 0.3 to 8 mass %, or from 0.5 to 8 mass %, or from 0.5 to 6 mass %, or from 0.8 to 5 mass %. When the content is the above-mentioned content, a powder preparation having excellent stability of zeaxanthin is more reliably formed.

In the present invention, the average particle diameter of the solid zeaxanthin is preferably from 0.03 to 2 μm, and may be from 0.03 to 1.5 μm, or from 0.05 to 1.2 μm, or from 0.05 to 1.0 μm, or from 0.08 to 1.0 μm, or from 0.05 to 0.8 μm, or from 0.05 to 0.6 μm, or from 0.05 to 0.5 μm, or from 0.1 to 0.6 μm, or from 0.1 to 0.5 μm. By setting the average particle diameter to the above-mentioned average particle diameter, a powder preparation having excellent stability of zeaxanthin is more reliably formed.

Further, in the powder preparation, the content ratio (mass % ratio) of the solid lutein to the solid zeaxanthin is preferably from 20:1 to 2:1, and the content ratio (mass % ratio) may be from 20:1 to 3:1, or from 20:1 to 4:1, or from 20:1 to 5:1, or from 19:1 to 2:1, or from 18:1 to 2:1, or from 18:1 to 3:1, or from 18:1 to 4:1, or from 18:1 to 5:1, or from 17:1 to 2:1, or from 17:1 to 3:1, or from 17:1 to 4:1, or from 17:1 to 5:1, or from 16:1 to 2:1, or from 15:1 to 2:1, or from 14:1 to 2:1, or from 13:1 to 2:1, or from 12:1 to 2:1, or from 11:1 to 2:1, or from 10:1 to 2:1, or from 9:1 to 2:1, or from 8:1 to 3:1, or from 7:1 to 4:1, or from 6:1 to 4:1, or from 6:1 to 5:1. When the content ratio of lutein to zeaxanthin is the above-mentioned ratio, a food or beverage which appeals to the functionality (for example, physiological activity) of lutein and zeaxanthin can be provided. Further, by setting the content ratio to the above-mentioned content ratio, it becomes easy to use the solid lutein and the solid zeaxanthin derived from a natural product such as an extract from marigold.

Further, in the powder preparation, the total content of the solid lutein and the solid zeaxanthin is preferably from 0.01 to 30 mass %, and may be from 0.02 to 25 mass %, or from 0.03 to 20 mass %, or from 0.05 to 18 mass %, or from 0.05 to 15 mass %, or from 0.05 to 12 mass %, or from 1 to 30 mass %, or from 3 to 25 mass %, or from 5 to 25 mass %, or from 7 to 25 mass %, or from 3 to 20 mass %, or from 4 to 20 mass %, or from 5 to 20 mass %, or from 7 to 20 mass %, or from 8 to 18 mass %, or from 3 to 15 mass %, or from 5 to 15 mass % in the powder preparation.

As the gum ghatti usable in the present invention, one descried for the solid dispersion composition can be appropriately used in the same manner.

Further, in the present invention, in the powder preparation, the content of the gum ghatti is preferably from 1 to 30 mass %, and may be from 2 to 25 mass %, or from 2.5 to 25 mass %, or from 3 to 25 mass %, or from 3.5 to 25 mass %, or from 4 to 25 mass %, or from 4.5 to 25 mass %, or from 5 to 25 mass %, or from 5 to 23 mass %. By setting the content to the above-mentioned content ratio, a powder preparation having excellent stability of lutein and zeaxanthin is more reliably formed.

Further, in the present invention, in the powder preparation, the content of the gum ghatti with respect to 1 part by mass of the total content of the solid lutein and the solid zeaxanthin is preferably from 0.1 to 15 parts by mass, and may be from 0.1 to 12 parts by mass, or from 0.1 to 10 parts by mass, or from 0.2 to 8 parts by mass, or from 0.2 to 7 parts by mass, or from 0.3 to 7 parts by mass, or from 0.2 to 5 parts by mass, or from 0.3 to 5 parts by mass, or from 0.3 to 5 parts by mass, or from 0.3 to 4 parts by mass, or from 0.3 to 3 parts by mass, or from 0.5 to 5 parts by mass, or from 0.5 to 4 parts by mass, or from 0.5 to 3 parts by mass. By setting the content to the above-mentioned content ratio, a powder preparation having excellent stability of lutein and zeaxanthin is more reliably formed.

As the water to be used in the present invention, any water usable in a food or beverage can be used without particular limitation.

Further, in the powder preparation, the content of the water is preferably from 0 to 10 mass %, and may be 1 mass % or more and less than 10 mass %, or from 1 to 9 mass %, or from 2 to 8 mass %, or from 3 to 7 mass %. By setting the content to the above-mentioned content ratio, a powder preparation according to the intended use can be prepared.

In the present invention, the powder preparation need only be in a form usable in the production or preparation of a food or beverage or the like, and includes a preparation in the form of a powder or a granule. The powder preparation of the present invention is a solid, and therefore has an excellent taste as compared with an emulsified preparation.

In the present invention, the powder preparation can be configured to have a pH less than 5 when a 1 mass % aqueous solution of the powder preparation is formed. By using the powder preparation, for example, it becomes possible to use it also in a food or beverage or the like having a lower pH, which had a problem that precipitation or aggregation is caused by acidification, or the like in the prior art. The pH of the powder preparation may be less than 5, or less than 4.5, or less than 4, or less than 3.8, or less than 3.5, or less than 3.3, or less than 3, and as the lower limit, for example, pH 1, pH 1.5, pH 2, pH 2.5, pH 2.8, or pH 3, or the like can be exemplified. By using the powder preparation of the present invention, for example, even in the case of a food or beverage or the like having a pH less than 3.8 (for example, a food or beverage or the like having a pH in the range where the lower limit is pH 1, pH 1.5, pH 2, pH 2.5, pH 2.8, pH 3, pH 3.3, pH 3.5, pH 3.7, or the like, and the upper limit is pH 3.7, pH 3.6, pH 3.5, pH 3.4, pH 3.3, pH 3.2, pH 3.1, pH 3.0, or the like, which are appropriately combined) which is impossible by the proposal of PTL 2 that the pH is set to a specific value of 3.8 to 4.8, a food or beverage or the like having excellent stability is obtained without causing precipitation, aggregation, or the like.

Further, in the powder preparation of the present invention, for example, a pH adjusting agent, a fragrance, a coloring agent, vitamin C (including a derivative), or the like, which is used in a food or beverage or the like, may be appropriately blended other than these.

The powder preparation of the present invention has the above-mentioned configuration, and therefore, a solid dispersion composition having excellent stability of lutein and zeaxanthin against light, and a powder preparation, and a food or beverage or the like having excellent dispersion stability of lutein and zeaxanthin after storage or the like can be obtained.

In addition, an emulsified preparation in the prior art indispensably requires a heating treatment for dissolving lutein and zeaxanthin in an oil phase, and therefore, deterioration such as cis-trans denaturation or thermal deterioration of the lutein or the zeaxanthin may be sometimes caused. However, the powder preparation of the present invention does not require a heating treatment or the like for dissolving lutein and zeaxanthin in an oil phase, or the like, and therefore, the powder preparation can be used without causing deterioration of the lutein or the zeaxanthin.

Further, the powder preparation of the present invention has the above-mentioned configuration, and therefore, the contents of lutein and zeaxanthin can be quantified. Accordingly, in a food or beverage using the powder preparation of the present invention, the accurate contents of lutein and zeaxanthin can be specified. This is a great advantage in a food or beverage which appeals to the functionality of lutein and zeaxanthin.

### <Method for Producing Solid Dispersion Composition and Powder Preparation>

The present invention relates to a method for producing a powder preparation including:

a step of preparing the solid dispersion composition; and

a step of powdering the solid dispersion composition.

Note that with respect to items that are not particularly described as a solid dispersion composition and a powder preparation, basically, the description of each component or the like used in the solid dispersion composition and the powder preparation in this specification shall be replaced similarly.

As for the powder preparation of the present invention, by using the production method including the step of preparing the solid dispersion composition and the step of powdering the solid dispersion composition, the powder preparation can be easily obtained, however, the production method is not limited to this production method.

In the step of preparing the solid dispersion composition, a known method may be used without particular limitation as long as each component for forming the solid dispersion composition is contained.

The solid dispersion composition can be prepared, for example, by mixing an aqueous solution containing gum ghatti with solid lutein and solid zeaxanthin.

For example, an aqueous solution (a) containing gum ghatti is prepared. To the aqueous solution (a), solid lutein and solid zeaxanthin are added and stirred, whereby an aqueous solution (b) is prepared.

The aqueous solution (b) may be provided as a solid dispersion composition as it is, or may be subjected to a homogenization treatment. Further, the aqueous solutions (a) and (b) may contain an arbitrary component such as a polyhydric alcohol such as glycerin, propylene glycol, and a sugar alcohol, and L-ascorbic acid, or citric acid (anhydrous).

The homogenization treatment is not particularly limited, and can be performed using, for example, an emulsifying/dispersing device such as a high-pressure homogenizer, a nanomizer, or a microfluidizer, and an ultrasonic disperser, or the like. The treatment conditions are also not particularly limited, and can be appropriately adjusted and carried out according to the formulation. For example, in the case of a high-pressure homogenizer, the conditions of 100 to 500 kg/cm², and 1 to 10 times, and the like are exemplified.

The stirring treatment is also not particularly limited, and for example, a conventional stirrer such as a propeller stirrer can be used.

When a solid dispersion composition containing solid lutein and solid zeaxanthin each having an average particle diameter of 0.03 to 2 μm is prepared, for example, a micronization treatment can be performed. In that case, the step of preparing the solid dispersion composition of the present invention can include a step of micronizing (pulverizing) solid lutein and solid zeaxanthin to a predetermined particle diameter or less.

The micronization treatment is not particularly limited and can be performed using a device (for example, a grinder) capable of reducing the average particle diameter of each of the solid lutein and the solid zeaxanthin to a predetermined value or less. For example, a micronization treatment using a wet grinder such as an Ultraviscomill, a Dyno-Mill, a Sandmill, or a CoBall Mill can be exemplified.

In the step of powdering the solid dispersion composition, a known method may be used without particular limitation.

As the step of powdering the solid dispersion composition, for example, a method in which solid lutein and solid zeaxanthin such as crystal lutein and crystal zeaxanthin are micronized by means of wet grinding or the like, followed by powdering by means of spray drying, freeze drying, or the like can be exemplified.

By using the method for producing a powder preparation of the present invention, a powder preparation having excellent stability of lutein and zeaxanthin against light when used in a food or beverage or the like, and the powder preparation having excellent dispersion stability of lutein and zeaxanthin after storage or the like can be easily obtained. In addition, for example, a fine powder preparation of the submicron order can be easily obtained.

Further, by using the method for producing a powder preparation of the present invention, the powder preparation capable of quantifying the contents of lutein and zeaxanthin can be easily obtained.

### <Food or Beverage or the Like>

The present invention relates to a food or beverage or the like containing the solid dispersion composition or the powder preparation.

Note that with respect to items that are not particularly described as a food or beverage or the like, basically, the description of each component or the like used in the solid dispersion composition and the powder preparation in this specification shall be replaced similarly.

For the food or beverage or the like of the present invention, a known method such as addition or mixing (in the form of a solution, a semi-solid, a solid, or the like) may be used without particular limitation as long as the solid dispersion composition, the powder preparation, or both are contained.

Examples of the food or beverage of the present invention include:

beverages such as drinks (for example, green tea, black tea, herbal tea, coffee, and cocoa), soft drinks (for example, carbonated drinks, fruit drinks, fruit juice drinks, vegetable drinks, coffee drinks, tea drinks, sports drinks, mineral water, near water, milk drinks, nutritional drinks, functional drinks, jelly drinks, non-alcoholic taste drinks, etc.), lactic acid bacteria drinks, and alcoholic drinks;

confectionery such as frozen sweets (for example, ice candy, ice cream, etc.), sugar confectionery (for example, candy, nougat, gummy, marshmallow, chewing gum, chocolate, etc.), patisseries (for example, cakes, cookies, macarons, jelly, pudding, Bavarian cream, etc.), snack confectionery, and Japanese confectionery (for example, dumplings, rice crackers, donuts, sponge cakes, etc.);

processed agricultural products such as dried vegetables and pickles;

processed marine products such as kamaboko (boiled fish cake);

noodles, cooked rice, and processed cereal products such as bread;

seasonings;

syrups, jams, etc.;

supplements, confectionery; and

processed meat products.

The food or beverage or the like of the present invention includes, other than foods and beverages, those obtained by appropriately adding, mixing, or the like the solid dispersion composition, the powder preparation, or both in a target material other than foods and beverages.

Examples of the food or beverage or the like include:

coloring agents, coloring additives;

cosmetics (for example, fragrance products such as perfumes, basic cosmetics (facial cleansing creams, vanishing creams, cleansing creams, cold creams, massage creams, milky lotions, face lotions, serums, facial packs, makeup removers, etc.), finishing cosmetics (foundations, talcum powders, lipsticks, lip balms, rouge, eyeliners, mascara, eye shadows, eyebrow pencils, eye packs, nail enamels, enamel removers, etc.), hair cosmetics (pomade, brilliantine, hair set lotions, hair sticks, solid hair dressings, hair oils, hair treatments, hair creams, hair tonics, hair liquids, hairsprays, bandoline, hair nourishing agents, hair dyes, etc.), suntan cosmetics (suntan products, sunscreen products, etc.), and medicated cosmetics (antiperspirants, aftershave lotions and gels, permanent wave agents, medicated soaps, medicated shampoos, medicated skin cosmetics, etc.));

aromatics (a solid type, a gel type, a liquid type, etc.);

toiletry products (for example, toilet soaps, bath soaps, perfume soaps, transparent soaps, synthetic soaps, etc.);

body cleansers (body soaps, body shampoos, hand soaps, etc.);

hair care products (shampoos, rinses, rinse-in shampoos, hair conditioners, hair treatments, hair packs, hair dyes, etc.);

skin care products (lip balms, hand creams), shaving products (shaving foams, etc.);

oral products (toothpastes, oral cleansers, mouthwashes, troches, chewing gum, etc.);

bath additives (bath salts, bath tablets, bath liquids, etc.), bath foams (bubble bath additives, etc.), bath oils (bath perfumes, bath capsules, etc.), milk bath additives, bath jellies, bath cubes, etc.;

pharmaceutical products; and

quasi-drugs.

Preferably, a food or beverage, a coloring agent, a coloring additive, a pharmaceutical product, or a quasi-drug is exemplified, and more preferably, a food or beverage, a coloring agent, or a coloring additive is exemplified, and a food or beverage is most preferred.

The content of the solid dispersion composition in the food or beverage or the like of the present invention is not particularly limited, however, for example, the total content of lutein and zeaxanthin in the food or beverage or the like can be from 0.00001 to 30 mass %, or from 0.0001 to 25 mass %, or from 0.001 to 20 mass %, or from 0.00001 to 1 mass %, or from 0.0001 to 1 mass %, or from 0.00001 to 0.1 mass %, or from 0.0001 to 0.1 mass %, or from 0.00001 to 0.01 mass %, or from 0.0001 to 0.01 mass %, or from 1 to 30 mass %, or from 1 to 25 mass %, or from 1 to 10 mass %. Further, when the food or beverage or the like is a beverage, for example, the total content of lutein and zeaxanthin in the beverage can be from 0.00001 to 0.1 mass %, or from 0.0001 to 0.05 mass %, or from 0.001 to 0.01 mass %.

In the present invention, the food or beverage or the like can be configured to have a pH less than 5. By using the food or beverage or the like, for example, it becomes possible to use it also in a food or beverage or the like having a lower pH, which had a problem that precipitation or aggregation is caused by acidification, or the like in the prior art. The pH of the food or beverage or the like may be less than 5, or less than 4.5, or less than 4, or less than 3.8, or less than 3.5, or less than 3.3, or less than 3, and as the lower limit, for example, pH 1, pH 1.5, pH 2, pH 2.5, pH 2.8, or pH 3, or the like can be exemplified. Further, when the food or beverage or the like contains water, in general, the pH measured as it is can be used, however, when appropriate measurement is difficult as it is, the pH when an aqueous solution having a solid content of 1 mass % is formed is used as a reference. By using the food or beverage or the like of the present invention, for example, even in the case of a food or beverage or the like having a pH less than 3.8 (for example, a food or beverage or the like having a pH in the range where the lower limit is pH 1, pH 1.5, pH 2, pH 2.5, pH 2.8, pH 3, pH 3.3, pH 3.5, pH 3.7, or the like, and the upper limit is pH 3.7, pH 3.6, pH 3.5, pH 3.4, pH 3.3, pH 3.2, pH 3.1, pH 3.0, or the like, which are appropriately combined) which is impossible by the proposal of PTL 2 that the pH is set to a specific value of 3.8 to 4.8, a food or beverage or the like having excellent dispersion stability is obtained without causing precipitation or aggregation, or the like.

Further, in the present invention, the food or beverage can be configured to have a pH more than 5. For example, the food or beverage of the present invention can have a pH more than 5, and the pH may be in the range of 5.1 to 7, or in the range of 5.3 to 6.9, or in the range of 5.5 to 6.5. According to the present invention, even in the above-mentioned pH range, a food or beverage having excellent stability of lutein and zeaxanthin can be provided.

Further, in the food or beverage or the like of the present invention, for example, a pH adjusting agent, a fragrance, a coloring agent, vitamin C (including derivative thereof), or the like, which is used in a food or beverage or the like, may be appropriately blended other than these.

By using the food or beverage or the like of the present invention, a food or beverage or the like having excellent stability of lutein and zeaxanthin against light, and a food or beverage or the like having excellent dispersion stability of lutein and zeaxanthin after storage or the like can be obtained.

Further, by using the food or beverage or the like of the present invention, a food or beverage or the like capable of quantitatively indicating the contents of lutein and zeaxanthin can be obtained.

Further, by using the food or beverage or the like of the present invention, a food or beverage or the like to which an indication regarding a function based on lutein and zeaxanthin is attached can be provided. For example, a food or beverage or the like to which an indication regarding a function based on adjustment of visual function, improvement of visual function, reduction of eye fatigue, an activity of supplementing the macular pigment of the eye (for example, an activity of maintaining or increasing the amount of the macular pigment, and an activity of maintaining or increasing the density of the macular pigment, etc.), improvement of sleep quality, improvement of neck stiffness, improvement of accuracy of memory, reduction of anti-cognitive function, maintenance or improvement of attention, or maintenance or improvement of flexibility of thought can be provided.

### EXAMPLES

Hereinafter, the present invention will be described with reference to Examples, however, the present invention is not limited to these Examples.

### Example 1

Based on the formulation in Table 1, the composition of Example 1 was prepared.

### (Preparation of Solid Dispersion Preparation)

Specifically, a liquid mixture was obtained by mixing lutein and zeaxanthin in a gum ghatti aqueous solution containing ion exchanged water, gum ghatti (“Gum Ghatti SD” manufactured by San-Ei Gen F.F.I., Inc.), L-ascorbic acid, citric acid (anhydrous), and propylene glycol. The liquid mixture was subjected to a micronization treatment with a wet grinder (a grinding treatment, “Dyno-Mill KDL (manufactured by WAB AG)”), followed by a homogenization treatment with a homogenizer (500 kg/cm²×5 times), whereby a composition of Example 1 was prepared. The average particle diameter of each of the solid lutein and the solid zeaxanthin in Example 1 was about 0.2 μm. Further, the pH of the solid dispersion composition of Example 1 was 2.76 (25.6° C.)

TABLE 1

| Formulation of solid dispersion composition | mass % |
|---|---|
| lutein, zeaxanthin | 1.32 |
| gum ghatti | 5.7 |
| L-ascorbic acid | 0.9 |
| citric acid (anhydrous) | 0.4 |
| ion exchanged water | 76.23 |
| propylene glycol | 15.45 |
| total | 100 |

### Test Example 1: Light Resistance Test

A light resistance test was performed for the solid dispersion composition prepared in Example 1 and lutein and zeaxanthin emulsified preparations distributed in the market.

### (Lutein and Zeaxanthin Samples Used)

Solid dispersion composition of Example 1

Lutein and zeaxanthin-containing emulsified preparation (1): lutein ester emulsified preparation

Lutein and zeaxanthin-containing emulsified preparation (2): lutein-free emulsified preparation

### (Formulation of Model Beverage)

A model beverage composed of fructose-glucose liquid sugar syrup (13.3 mass %), citric acid (anhydrous) (0.2 mass %), trisodium citrate (0.08 mass %), L-ascorbic acid (0.02 mass %), each of the lutein and zeaxanthin samples (the total amount of lutein and zeaxanthin: 20 ppm), and ion exchanged water (balance) was prepared (pH: 3.2, Brix: 10°).

### (Irradiation Test)

Each of the above model beverages was filled in a 50-mL screw bottle, and an irradiation test was performed under the following irradiation conditions (irradiation dose: 1000 Langley).

### (Irradiation Conditions)

Device: Xenon Long Light Fade Meter XML-75R

Intensity: 600 W/m²

Time: 20 hours

Temperature: 20° C.

Humidity: 50%

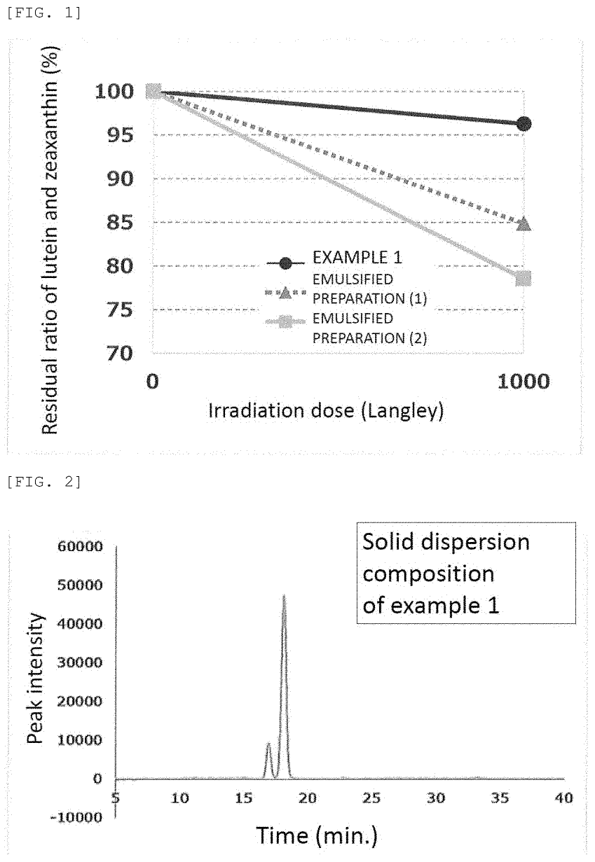

The contents of lutein and zeaxanthin before and after the irradiation test were measured, and the residual ratio of lutein and zeaxanthin were calculated. The results are shown in Table 2 and FIG. 1.

### (Measurement of Residual Ratio of Lutein and Zeaxanthin)

To 10 g of each of the model beverages before and after irradiation, 40 mL of 99.5% ethanol was added, followed by sonication for 10 minutes, and the volume was increased to 50 mL with 99.5% ethanol. The volume of the solution (10 mL) was increased to 20 mL with 99.5% ethanol, followed by filtration through a 0.2 μm PTFE filter, and the absorbance of the filtrate at the maximum absorption wavelength (around 450 nm) was measured.

The total content of lutein and zeaxanthin was calculated from the following Formula 1.

Further, the residual ratio (%) was calculated from the following Formula 2.

Total content of lutein and zeaxanthin (ppm)=absorbance×50×2×10000/2550×weight (g) (Formula 1)

Residual ratio (%)=(total content of lutein and zeaxanthin after light irradiation/total content of lutein and zeaxanthin before light irradiation)×100 (Formula 2)

TABLE 2

| | Residual |
|---|---|
| | ratio % |
| solid dispersion composition of Example 1 | 96 |
| lutein and zeaxanthin-containing emulsified preparation (1) | 85 |
| lutein and zeaxanthin-containing emulsified preparation (2) | 79 |

From Table 2 and FIG. 1, it was indicated that the solid dispersion composition of the present invention has excellent light resistance.

### Test Example 2: Quantitative Test

An HPLC analysis was performed for each of the lutein and zeaxanthin samples used in the above Test Example 1. The results are shown in FIGS. 2 to 4. In addition, the HPLC analysis conditions are as follows.

### (HPLC Analysis Conditions)

Detector: ultraviolet-visible absorptiometer (measurement wavelength: 450 nm)

Column: 4.6×250 mm ODS

Column temperature: 50° C.

Mobile phase: methanol/tetrahydrofuran/ultrapure water=45/30/25

Flow rate: 1.0 mL/min

Injection volume: 30 μL

Analysis time: 30 min

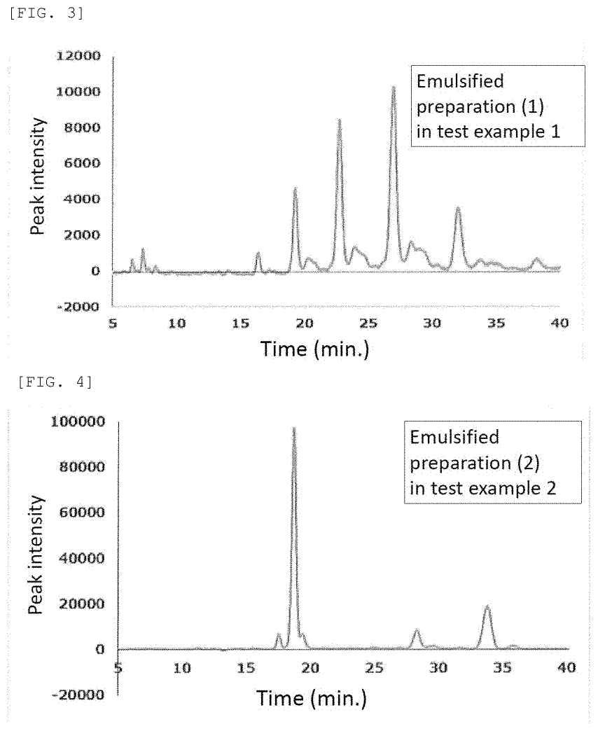

As shown in FIGS. 3 and 4, it was indicated that in the lutein and zeaxanthin emulsified preparations distributed in the market, it is difficult to quantify lutein and zeaxanthin. In addition, from FIG. 4, it is inferred that the “lutein and zeaxanthin-containing emulsified preparation (2)” is partially converted to cis-form.

On the other hand, as shown in FIG. 2, in the solid dispersion composition of Example 1, lutein and zeaxanthin can be quantified, and the content ratio thereof was as follows: lutein:zeaxanthin=5:1.

### Test Example 3: Storage Test

Lutein and zeaxanthin-containing solid dispersion compositions (Comparative Examples 1 to 4) were prepared in the same manner as the solid dispersion composition of Example 1 except that gum ghatti was replaced with modified starch or any of various emulsifiers.

### (Materials Used)

Comparative Example 1: modified starch (starch sodium octenyl succinate)

Comparative Example 2: polyglycerin fatty acid ester (1): decaglycerin monooleate

Comparative Example 3: polyglycerin fatty acid ester (2): decaglycerin monostearate

Comparative Example 4: sucrose fatty acid ester: sucrose palmitate

### (Evaluation of Dispersibility)

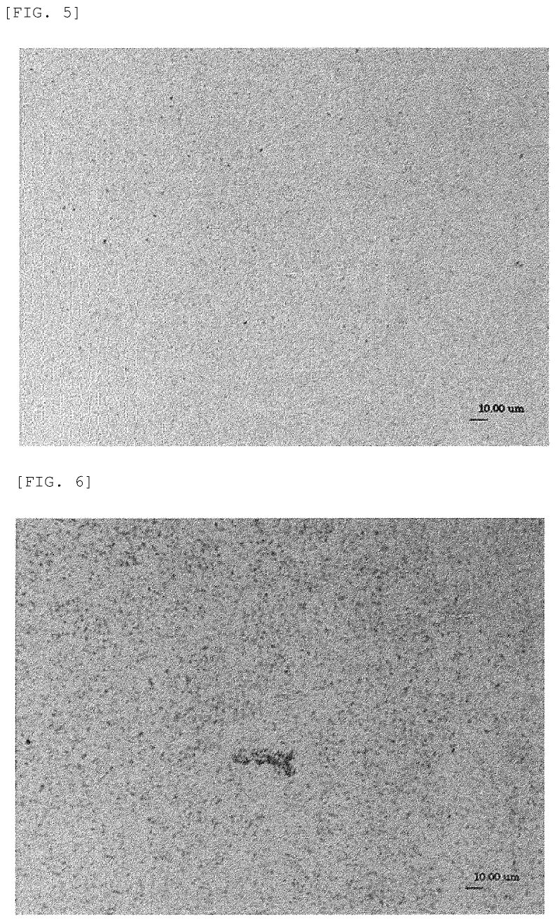

The compositions obtained in Example 1 and Comparative Examples 1 to 4 were stored at 60° C. for 7 days, and the results of evaluating the dispersibility of lutein and zeaxanthin after the storage are shown in Table 3 and FIGS. 5 and 6.

### (Evaluation Method)

D50 μm: median diameter (μm)

Particle variation ratio: median diameter after storage/median diameter before storage×100(%)

1.3 μm ↑: frequency of particle diameters when the particle diameter is 1.3 μm or more (%)

With or without aggregation: It was confirmed whether aggregation of particles occurred by optical microscopic observation (600 times (eyepiece 15 times, objective 40 times)).

With respect to the median diameter or the frequency of particle diameters, the particle size distribution of the solid dispersion composition was measured under the following conditions.

### (Measurement Conditions)

Particle size distribution analyzer: Microtrac MT3000EX-II (manufactured by MicrotracBEL Corporation)

Measurement method: refractive index: 1.81, measurement range: from 0.021

TABLE 3

| | | D50 μm (μm) | | Variation ratio | 1.3 μm or more (%) | | |
|---|---|---|---|---|---|---|---|
| | | Before | After | of particle | Before | After | with or without |
| | Material used | storage | storage | diameter (%) | storage | storage | aggregation |
| Example 1 | gum ghatti | 0.23 | 0.23 | 100 | 0 | 0 | without (FIG. 5) |
| Comparative | modified starch | 0.29 | 0.37 | 128 | 1 | 8 | with aggregation |
| Example 1 | | | | | | | |
| Comparative | polyglycerin fatty | 0.32 | 0.42 | 131 | 1 | 10 | with significant |
| Example 2 | acid ester (1) | | | | | | aggregation on the whole |
| | | | | | | | (FIG. 6) |
| Comparative | polyglycerin fatty | 0.27 | 0.40 | 148 | 1 | 9 | with significant |
| Example 3 | acid ester (2) | | | | | | aggregation on the whole |

| | | |
|---|---|---|
| Comparative | sucrose fatty acid | aggregation occurred significantly, and preparation could not be made |
| Example 4 | ester | |

As shown in Table 3 and FIG. 5, when the composition of Example 1 was used, no aggregation occurred during storage, and the composition had excellent dispersibility. In addition, the particle diameters of the solid lutein and the solid zeaxanthin did not vary before and after the storage, and the composition had excellent stability.

On the other hand, it was found that when the compositions of Comparative Examples 1 to 4 using the modified starch or the surfactant in place of the gum ghatti of Example 1 were used, in the case of Comparative Examples 1 to 3, aggregation occurred during storage, and the D50 μm value also increased from the beginning, and the stability of the particle size was poor. Aggregation causes uneven distribution of the solid lutein and the solid zeaxanthin in the solid dispersion composition, and hinders accurate quantification of lutein and zeaxanthin. From this viewpoint as well, it was indicated that the compositions of Comparative Examples 1 to 3 are undesirable. Further, in the case of Comparative Example 4, preparation was attempted, but aggregation occurred significantly and preparation could not be made.

### Test Example 4: Long-Term Storage Test

The solid dispersion composition of Example 1 was subjected to a long-term storage test.

A model beverage was prepared using the solid dispersion composition of Example 1, and the model beverage was stored under the condition of 40° C. for 2 months, and then, the content of each of lutein and zeaxanthin before and after the storage was measured, and the residual ratio was calculated. The contents of lutein and zeaxanthin were measured by an HPLC analysis under the same conditions as in the quantitative test of Test Example 2.

### (Model Beverage)

A model beverage composed of fructose-glucose liquid sugar syrup (13.3 mass %), the composition of Example 1 (0.23 mass %), citric acid (anhydrous) (0.2 mass %), trisodium citrate (0.08 mass %), L-ascorbic acid (0.02 mass %), and ion exchanged water (balance) was prepared. Further, the model beverage had a pH of 3.0 and a Brix of 10°.

### (Results)

The residual ratios of lutein and zeaxanthin after the storage at 40° C. for 2 months were 99.2% and 95.8%, respectively. Further, in the solid dispersion composition of Example 1, no precipitate or aggregate was observed.

From these results, it was indicated that the solid dispersion composition of the present invention has high stability of lutein and zeaxanthin even during long-term storage.

### Test Example 5: Light Resistance Test

A light resistance test was performed for a model beverage having the same formulation as in Test Example 4. The contents of lutein and zeaxanthin before and after irradiation were measured by an HPLC analysis, and the residual ratios of lutein and zeaxanthin were calculated.

(Irradiation conditions) same as in Test Example 1

(HPLC analysis conditions) same as in Test Example 2

### (Results)

The residual ratios of lutein and zeaxanthin after the light resistance test were 96% and 95.8%, respectively, and high residual ratios were exhibited.

### Example 2

Based on the formulation in Table 4, solid dispersion compositions of Examples 2-1 to 2-4 were prepared (liquid preparations).

### (Preparation of Solid Dispersion Preparation)

A liquid mixture was obtained by adding a mixture (solid) of lutein and zeaxanthin to an aqueous solution containing ion exchanged water, gum ghatti, refined salt, L-ascorbic acid, citric acid (anhydrous), and propylene glycol. The liquid mixture was subjected to a micronization treatment with a grinder (a grinding treatment, “Dyno-Mill KDL (manufactured by WAB AG)”), followed by a homogenization treatment with a homogenizer (500 kg/cm²×5 times), whereby the compositions of Examples 2-1 to 2-4 were prepared. The contents of lutein and zeaxanthin are shown in Table 4. Note that the pH of the solid dispersion compositions of Examples 2-1 to 2-4 was in the range of 2.5 to 2.9.

TABLE 4

| Formulation of solid dispersion | | | | |
|---|---|---|---|---|
| composition (mass %) | Example 2-1 | Example 2-2 | Example 2-3 | Example 2-4 |
| lutein and zeaxanthin mixture A | 1.32 | — | 1.32 | 1.32 |
| lutein and zeaxanthin mixture B | — | 1.32 | — | — |
| gum ghatti | 5.7 | 5.7 | 11.38 | 3 |
| refined salt | 4.5 | 4.5 | 4.5 | 4.5 |
| L-ascorbic acid | 0.9 | 0.9 | 0.9 | 0.9 |
| citric acid (anhydrous) | 0.4 | 0.4 | 0.4 | 0.4 |
| ion exchanged water | 72.18 | 72.18 | 66.5 | 74.88 |
| propylene glycol | 15 | 15 | 15 | 15 |
| total | 100 | 100 | 100 | 100 |
| lutein content | 1.11 | 1.24 | 1.11 | 1.11 |
| zeaxanthin content | 0.21 | 0.08 | 0.21 | 0.21 |

### Test Example 5: Storage Test

A storage test was performed for the prepared solid dispersion compositions of Examples 2-1 to 2-4 in the same manner as in Test Example 3.

The solid dispersion compositions of Examples 2-1 to 2-4 were stored at 60° C. for 7 days, and thereafter, the dispersibility of lutein and zeaxanthin after the storage were evaluated. The results are shown in Table 5.

TABLE 5

| | D50 μm (μm) | | | | |
|---|---|---|---|---|---|
| | | | Variation ratio | 1.3 μm or more (%) | |
| | Before | After | of particle | Before | After |
| | storage | storage | diameter (%) | storage | storage |
| Example 2-1 | 0.22 | 0.22 | 100% | 0 | 0 |
| Example 2-2 | 0.21 | 0.22 | 105% | 0 | 0 |
| Example 2-3 | 0.22 | 0.22 | 100% | 0 | 0 |
| Example 2-4 | 0.22 | 0.24 | 109% | 0 | 0 |

As shown in Table 5, it was confirmed that in all the solid dispersion compositions of Examples 2-1 to 2-4, the particle diameters of the solid lutein and the solid zeaxanthin did not vary before and after the storage, and the compositions have excellent stability.

### Test Example 6: Light Resistance Test

A light resistance test was performed for the solid dispersion composition prepared in Example 2-1.

### (Formulation of Model Beverage)

Model Beverage with pH of 3.0

A model beverage composed of fructose-glucose liquid sugar syrup (13.3 mass %), citric acid (anhydrous) (0.2 mass %), trisodium citrate (0.07 mass %), L-ascorbic acid (0.02 mass %), the solid dispersion preparation of Example 2-1 (0.2 mass %), and ion exchanged water (balance) was prepared. In the test, one obtained by hot-packing the model beverage in a 500-mL plastic bottle at 93° C. was used.

Model Beverage with pH of 6.3

A model beverage composed of fructose-glucose liquid sugar syrup (13.3 mass %), citric acid (anhydrous) (0.2 mass %), trisodium citrate (3.7 mass %), L-ascorbic acid (0.02 mass %), the solid dispersion preparation of Example 2-1 (0.2 mass %), and ion exchanged water (balance) was prepared. In the test, one obtained by hot-packing the model beverage in a 500-mL plastic bottle at 93° C. was used.

### (Light Resistance Test)

A light resistance test was performed for each of the model beverages packed in the 500-mL plastic bottle.

Device: Xenon Long Light Fade Meter XML-75R (manufactured by Suga Test Instruments Co., Ltd.)

The contents of lutein and zeaxanthin before and after the light resistance test were measured, and the residual ratios of lutein and zeaxanthin were calculated. The results are shown in Table 6. The measurement of the residual ratios of lutein and zeaxanthin was performed in the same manner as in Test Example 1.

TABLE 6

| pH of | | Before test | 500 Langley | Residual | 1000 Langley | Residual |
|---|---|---|---|---|---|---|
| beverage | | (mg/bottle) | (mg/bottle) | ratio (%) | (mg/bottle) | ratio (%) |
| 3.0 | lutein content | 10.6 | 9.3 | 87.7 | 8.7 | 82.1 |
| | zeaxanthin content | 2.0 | 1.6 | 80.0 | 1.5 | 75.0 |
| 6.3 | lutein content | 10.8 | 10.5 | 97.2 | 10.4 | 96.3 |
| | zeaxanthin content | 1.9 | 1.9 | 100.0 | 1.9 | 100.0 |

From Table 6, it was indicated that the solid dispersion composition of the present invention has excellent light resistance in a pH range of either of pH 3.0 and pH 6.3.

### Test Example 7: Heat Resistance Test

A heat resistance test was performed for the solid dispersion composition prepared in Example 2-1. As a model beverage, the same one as in Test Example 6 was used.

### (Heat Resistance Test)

A heat resistance test was performed for each of the above model beverages packed in the 500-mL plastic bottle. Before and after the heat resistance test, visual observation and microscopic observation were performed, and the turbidity and the contents of lutein and zeaxanthin were measured.

Conditions: See Table 7

Turbidity: For the model beverage, the absorbance at 720 nm was measured using the following device and cell.

Device: spectrophotometer V-550 (manufactured by JASCO Corporation)

Cell: quartz cell, 10 mm×10 mm

Contents of lutein and zeaxanthin: measured in the same manner as in Test Example 1

The conditions for the heat resistance test for the model beverage with a pH of 3.0 and the results are shown in Table 7, and the conditions for the heat resistance test for the model beverage with a pH of 6.3 and the results are shown in Table 8.

TABLE 7

| | Model beverage with | | | | | |
|---|---|---|---|---|---|---|
| Temperature | pH of 3.0 | Before test | 30 sec | 1 month | 2 months | 6 months |
| 25° C. | visual observation | no abnormality | — | no abnormality | no abnormality | no abnormality |
| | microscopic observation | no abnormality | — | no abnormality | no abnormality | no abnormality |
| | turbidity | 0.199 | — | 0.194 | 0.194 | 0.193 |
| | lutein content | 10.9 | — | 10.8 | 10.9 | 10.8 |
| | zeaxanthin content | 2.0 | — | 1.9 | 1.8 | 1.9 |
| 40° C. | visual observation | no abnormality | — | no abnormality | no abnormality | — |
| | microscopic observation | no abnormality | — | no abnormality | no abnormality | — |
| | turbidity | 0.199 | — | 0.194 | 0.194 | — |
| | lutein content | 10.9 | — | 10.9 | 10.5 | — |
| | zeaxanthin content | 2.0 | — | 2.0 | 1.6 | — |
| 90° C. | lutein content | 10.8 | 10.9 | — | — | — |
| | zeaxanthin content | 2.0 | 2.0 | — | — | — |
| 120° C. | lutein content | 10.8 | 10.7 | — | — | — |
| | zeaxanthin content | 2.0 | 2.0 | — | — | — |
| 140° C. | lutein content | 10.8 | 10.6 | — | — | — |
| | zeaxanthin content | 2.0 | 1.9 | — | — | — |

In the table, the lutein content and the zeaxanthin content mean (mg/bottle).

TABLE 8

| | Model beverage with | | | | | |
|---|---|---|---|---|---|---|
| Temperature | pH of 6.3 | Before test | 30 sec | 1 month | 2 months | 6 months |
| 25° C. | visual observation | no abnormality | — | no abnormality | no abnormality | no abnormality |
| | microscopic observation | no abnormality | — | no abnormality | no abnormality | no abnormality |
| | turbidity | 0.200 | — | 0.196 | 0.193 | 0.198 |
| | lutein content | 10.8 | — | 11.2 | 10.9 | 10.9 |
| | zeaxanthin content | 1.9 | — | 2.0 | 1.9 | 1.9 |
| 40° C. | visual observation | no abnormality | — | no abnormality | no abnormality | — |
| | microscopic observation | no abnormality | — | no abnormality | no abnormality | — |
| | turbidity | 0.200 | — | 0.196 | 0.206 | — |
| | lutein content | 10.8 | — | 10.7 | 10.9 | — |
| | zeaxanthin content | 1.9 | — | 1.9 | 1.9 | — |
| 90° C. | lutein content | 10.7 | 10.8 | — | — | — |
| | zeaxanthin content | 2.0 | 1.9 | — | — | — |
| 120° C. | lutein content | 10.7 | 10.6 | — | — | — |
| | zeaxanthin content | 2.0 | 1.9 | — | — | — |
| 140° C. | lutein content | 10.7 | 10.4 | — | — | — |
| | zeaxanthin content | 2.0 | 1.9 | — | — | — |

In the table, the lutein content and the zeaxanthin content mean (mg/bottle).

As shown in Tables 7 and 8, the solid dispersion composition of the present invention exhibited high stability also in the long-term storage test and the high-temperature test at 90° C. or higher.

### Test Example 8: Shake Resistance Test

A shake resistance test was performed for the solid dispersion composition prepared in Example 2-1. As a model beverage, the same one as in Test Example 6 was used. The results are shown in Table 9.

### (Shake Resistance Test)

Shaking test: amplitude: 2 cm, 140 strokes/min, 12 hours

Device: shaker NR-150 (manufactured by Taitec Corporation)

Transport test: The prepared model beverage was transported three times back and forth between Tokyo and Osaka by a truck.

TABLE 9

| pH of model | | | | |
|---|---|---|---|---|
| beverage | | Before test | Shaking test | Transport test |
| pH 3.0 | visual observation | no abnormality | no abnormality | no abnormality |
| | microscopic observation | no abnormality | no abnormality | no abnormality |
| pH 6.3 | visual observation | no abnormality | no abnormality | no abnormality |
| | microscopic observation | no abnormality | no abnormality | no abnormality |

As shown in Table 9, it was confirmed that in the solid dispersion composition of the present invention, aggregation did not occur even when an impact by shaking was applied, and the composition has high stability.

### Example 3

Based on the formulation in Table 10, a solid dispersion composition of Example 3 was prepared (powder preparation).

### (Preparation of Spray Liquid)

A gum ghatti aqueous solution was prepared by heating a mixture of gum ghatti, citric acid, maltose, dextrin, and water to 90° C., followed by cooling to 60° C. or lower. The resulting aqueous solution, a mixture of lutein and zeaxanthin, ethanol, and ascorbic acid were mixed, followed by a micronization treatment with a grinder (a grinding treatment, “Dyno-Mill KDL (manufactured by WAB AG)”), and then, a homogenization treatment with a homogenizer, whereby a spray liquid was obtained (500 kg/cm²×5 times). The obtained spray liquid was subjected to a powdering treatment by spray drying, whereby the solid dispersion composition (powder preparation) of Example 3 was prepared. The formulation of the powder preparation after powdering is shown in Table 11.

TABLE 10

| Formulation of spray liquid (mass %) | Example 3 |
|---|---|
| mixture of lutein and zeaxanthin | 4.08 |
| gum ghatti | 5.07 |
| citric acid | 0.16 |
| maltose | 3.69 |
| dextrin | 10.63 |
| L-ascorbic acid | 1.50 |
| ethanol | 3.91 |
| ion exchanged water | 70.96 |
| total | 100 |

TABLE 11

| Formulation of powder preparation (mass %) | Example 3 |
|---|---|
| mixture of lutein and zeaxanthin | 16.2 |
| gum ghatti | 20.17 |
| citric acid | 0.62 |
| maltose | 14.68 |
| dextrin | 42.35 |
| L-ascorbic acid | 5.98 |
| total | 100 |
| lutein content | 11.2 |
| zeaxanthin content | 2.2 |

### Test Example 9: Storage Test

A storage test was performed for the spray liquid before powdering and the prepared solid dispersion composition of Example 3.

The spray liquid before powdering was stored at 60° C. for 1 day, and the median diameter (D50 μm) and the frequency of particle diameters when the particle diameter is 1.3 μm or more (1.3 μm ↑/%) before and after the storage were measured. The measurement was performed using the same device and conditions as in Test Example 3.

Similarly, the solid dispersion composition of Example 3 was stored at 60° C. for 7 days, and the median diameter (D50 μm/μm) and the frequency of particle diameters when the particle diameter is 1.3 μm or more (1.3 μm ↑/%) before and after the storage were measured. The measurement of the median diameter or the frequency of particle diameters was performed for a solution in which the powder was diluted to 10% with ion exchanged water.

The results are shown in Table 12

TABLE 12

| | D50μm (μm) | | | | |
|---|---|---|---|---|---|
| | | | Variation ratio | 1.3 μm or more (%) | |
| | Before | After | of particle | Before | After |
| | storage | storage | diameter (%) | storage | storage |
| Spray liquid | 0.23 | 0.23 | 100% | 2.2 | 5.4 |
| Example 3 | 0.28 | 0.31 | 111% | 2.2 | 3.3 |

As shown in Table 12, it was confirmed that in the solid dispersion composition of Example 3, the particle diameters of the solid lutein and the solid zeaxanthin did not vary much before and after the storage, and the composition has excellent stability.

1. A solid dispersion composition, comprising solid lutein, solid zeaxanthin, gum ghatti, and water.

2. The solid dispersion composition according to claim 1, wherein in the solid dispersion composition, the content ratio (mass % ratio) of the solid lutein to the solid zeaxanthin is from 20:1 to 2:1.

3. The solid dispersion composition according to claim 1, wherein the average particle diameter of each of the solid lutein and the solid zeaxanthin is from 0.03 to 2 μm.

4. The solid dispersion composition according to claim 1, wherein in the solid dispersion composition, the total content of the solid lutein and the solid zeaxanthin is from 0.1 to 30 mass %.

5. The solid dispersion composition according to claim 1, wherein the content of gum ghatti with respect to 1 part by mass of the total content of the solid lutein and the solid zeaxanthin is from 0.1 to 15 parts by mass.

6. The solid dispersion composition according to claim 1, wherein the pH is less than 5.

7. A method for producing a powder preparation, comprising:
preparing the solid dispersion composition according to claim 1; and
powdering the solid dispersion composition.

8. A powder preparation, comprising solid lutein, solid zeaxanthin, and gum ghatti.

9. The powder preparation according to claim 8, wherein in the powder preparation, the content ratio (mass % ratio) of the solid lutein to the solid zeaxanthin is from 20:1 to 2:1.

10. The powder preparation according to claim 8, wherein the average particle diameter of each of the solid lutein and the solid zeaxanthin is from 0.03 to 2 μm.

11. The powder preparation according to claim 8, wherein the content of gum ghatti with respect to 1 part by mass of the total content of the solid lutein and the solid zeaxanthin is from 0.1 to 15 parts by mass.

12. The powder preparation according to claim 8, wherein the pH when a 1 mass % aqueous solution of the powder preparation is formed is less than 5.

13. A food or beverage, a coloring agent, a coloring additive, a cosmetic, an aromatic, a body cleanser, a hair care product, a skin care product, an oral product, a bath additive, a pharmaceutical product, or a quasi-drug, comprising the solid dispersion composition according to claim 1.

14. The food or beverage, the coloring agent, the coloring additive, the cosmetic, the aromatic, the body cleanser, the hair care product, the skin care product, the oral product, the bath additive, the pharmaceutical product, or the quasi-drug according to claim 13, wherein the pH is less than 3.8.

15. A food or beverage, a coloring agent, a coloring additive, a cosmetic, an aromatic, a body cleanser, a hair care product, a skin care product, an oral product, a bath additive, a pharmaceutical product, or a quasi-drug, comprising the powder preparation according to claim 8.
